(19) United States
(12) Patent Application Publication
(10) Pub. No.: US 2025/0375259 A1
(43) Pub. Date: Dec. 11, 2025

(54) BILATERAL SURGICAL PLATFORM

(71) Applicant: GSB LLC, Nashville, TN (US)

(72) Inventors: John Michael Fitzpatrick, Nashville, TN (US); Gregoire Samuel Blachon, Nashville, TN (US)

(21) Appl. No.: 19/233,434

(22) Filed: Jun. 10, 2025

Related U.S. Application Data
(60) Provisional application No. 63/658,422, filed on Jun. 10, 2024.

(51) Int. Cl. A61B 90/11; A61B 90/10; A61B 90/50

(52) U.S. Cl. CPC A61B 90/11; A61B 90/50; A61B 2090/103

(57) ABSTRACT

The disclosure is directed to a Bilateral Surgical Platform that is configured to simultaneously align two surgical instruments along different trajectories during surgery including two planar bodies wherein each planar body includes a hole, two couplers wherein one coupler is inserted into the hole in one of the planar bodies and the other coupler is inserted into the hole in the other planar body, a connector wherein the connector is attached to each planar body, and further wherein each planar body comprises one or several legs with each leg configured to attach to an anchor implanted in a patient's head.

### CROSS REFERENCE TO RELATED APPLICATIONS

This application claims priority to U.S. Provisional Application No. 63/658,422 filed on Jun. 10, 2024, which is herein incorporated by reference in its entirety.

### BACKGROUND

### Technical Field of the Invention

The technical field of the invention is surgical instrumentation, and more specifically, a surgical platform that can align surgical instruments along trajectories during surgery.

### Description of the Related Art

Some surgical procedures rely on precisely guiding an instrument into the body, for example, the brain. This is the case in Stereotactic Surgery in which a target point within a brain is identified in a three-dimensional Scanned image of the body. Head frames have traditionally been used in this type of surgery, but they are large, heavy, uncomfortable, and frightening to patients, and they cannot simultaneously guide two instruments along differing trajectories for bilateral electrode insertions.

Rigid, custom-shaped platforms that mount onto tiny skull-implanted anchors that serve as fiducial markers have been recently introduced and are far smaller and lighter than a frame. They never need to be worn by the patient outside the operating room and require no adjustment. Furthermore, they have better accuracy than a frame.

### SUMMARY DISCLOSURE OF THE INVENTION

The disclosure is accordingly directed to a bilateral surgical platform that is configured to simultaneously align two surgical instruments along different trajectories during surgery including two planar bodies, wherein each planar body includes a hole, two couplers wherein one coupler is inserted into the hole in one of the planar bodies and the other coupler is inserted into the hole in the other planar body, a connector wherein the connector is attached to each planar body, and further wherein each planar body includes one or several legs with each leg configured to attach to an anchor implanted in a patient's head.

The bilateral surgical platform can have the two planar bodies including a thermoplastic polymer or a metal, the two couplers including a thermoplastic polymer or a metal, the connector including a thermoplastic polymer or a metal, and each leg including a thermoplastic polymer or a metal. The bilateral surgical platform can include the two planar bodies including polycarbonate, polyetherimide (Ultem™), polyether ether ketone (PEEK™), aluminum, stainless steel, or titanium; the two couplers including polycarbonate, Ultem™, PEEK™, aluminum, stainless steel, or titanium; the connector including polycarbonate, Ultem™, PEEK™, aluminum, stainless steel, or titanium; and each leg including polycarbonate, Ultem™, PEEK™, aluminum, stainless steel, or titanium.

The bilateral surgical platform can have the two planar bodies including polycarbonate; the two couplers including polycarbonate; the connector including aluminum; and each leg including stainless steel or titanium. The bilateral surgical platform can include the anchor including titanium. The bilateral surgical platform can include each planar body having two legs. The bilateral surgical platform can include one planar body including one leg and the other planar body including two legs.

A method of making the bilateral surgical platform includes implanting a plurality of anchors into a patient's head, taking a Computed Tomography Scan (CT) of a patient's head including the anchors, wherein the computed tomography scan includes a coordinate system, determining coordinates for the position and orientation for each anchor, for each entry point and target point on the patient's head, the anterior commissure, and the posterior commissure, determining, using the coordinates, with a manufacturing computer program, the shapes of the two planar bodies, the positions of the holes in the two planar bodies, and the lengths of the legs, manufacturing the planar bodies, and manually assembling the bilateral surgical platform. The method can include the computer program including a three-degree-of-freedom Computer-Numeric-Control (CNC) router.

In further embodiments, the disclosure is directed to a surgical platform for engaging with two surgical instruments to align each instrument with a trajectory of interest in a living subject comprising the following members, each made of a hard, heat-resistant material, such as polycarbonate, Ultem™, PEEK™, aluminum, stainless steel, or titanium including a first custom-shaped body member with a planar bottom surface and a top surface with two sections, the first section having a planar surface parallel to the bottom surface with through holes with countersinks—one shaped to fit a coupling member and two shaped to fit leg members—and the second section comprising a set of steps, also referred to as “stairsteps”, of equal depths with two through holes, each shaped to fit a bolt on the connecting member; a second body member of the same material as the first, with the same description as the first, but likely to have a different custom shape from the first; a rectangular shaped connecting member having parallel top and bottom planar surfaces with two sections, each with two through holes, each hole of which has a partially hemispherical upper portion and a conical lower portion, and four bolts, one in each hole with a spherical upper portion of the bolt mating with the partially hemispherical upper portion of the hole and a threaded shaft extending through the conical lower portion of the hole and into a hole in the second section of one of the body members and tightened with a nut onto the body member such that a body member is tightened onto each section of the connecting member, thus rigidly fastening the two body members together; two coupling members shaped to engage with a surgical instrument, each of which is inserted into the properly shaped countersink and through hole in one of the body members and attached rigidly to the body member with screws; four leg members, each with a head at the top and shaft that fits a countersink and through hole in a body member, each inserted through a hole, each with a rod extending through a cylindrical hole in the leg with male threads at its top that fit female threads in the head and a conical shaped end, which when the rod is twisted clockwise with a hex wrench inserted into a hexagonal depression at the top moves the rod toward the bottom, and the each with following features for its shaft: threads extending immediately below the head serving to tighten the leg rigidly to the body member with a nut, a spherical depression at the bottom, two grippers on opposite sides of bottom, which when twisting the rod moves it toward the bottom, are pushed together to grip a sphere.

The surgical platform can further have a connecting member that is bent so that its two sections are angled relative to each other. The surgical platform can also have three leg members instead of four and a maximum of two in either body member.

The surgical platform can further include an extender, made of titanium, which has a male-threaded bottom section that can be screwed into a bone-implanted anchor and a spherical upper section, which can be gripped by the grippers.

The surgical platform can further include a countersink of each leg hole oriented so that the grippers of the leg that is inserted in the hole will not collide with an extender when gripping the spherical portion of the extender regardless of the angle between the extender and the leg shaft.

The surgical platform can further include having the rod in the leg for pushing the grippers together replaced by a hollow cylinder near the bottom of the leg with inner female threads that match external male threads on the leg and which when twisted in one direction moves down to push the grippers together to grip the sphere and when twisted in the other direction moves up to release the grip of the sphere.

The surgical platform can further include having the rod in the leg for pushing the grippers together replaced by splitting the bottom end of the leg into a middle section and two outer sections shaped so that they can be positioned with the middle section sitting on top of a sphere, and the outer sections at the sides of the sphere. In addition, a hollow cylinder surrounds the leg near the bottom of the leg with inner female threads that match external male threads on the leg, which when twisted in one direction moves down to tighten the left and right sections of the leg together onto the sphere and when twisted in the other direction moves up to release their grip of the sphere.

Other features and aspects will be apparent from the following detailed description, the drawings, and the claims.

### BRIEF DESCRIPTION OF THE DRAWINGS

FIG. 1 shows an embodiment of the invention.

FIG. 2 shows another embodiment of the invention.

FIG. 3 shows another embodiment of the invention.

FIG. 4 shows an embodiment of a planar body of the invention.

FIG. 5 shows an embodiment of stair steps of the invention.

FIG. 6 shows embodiments of a connector of the invention.

FIG. 7 shows embodiments of legs of the invention.

FIG. 8 shows embodiments of connectors of the invention.

FIG. 9 shows embodiments of legs of the invention.

FIG. 10 shows embodiments of legs of the invention.

FIG. 11 shows embodiments of legs of the invention.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DISCLOSURE OF THE INVENTION

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

### Modes for Carrying out the Invention

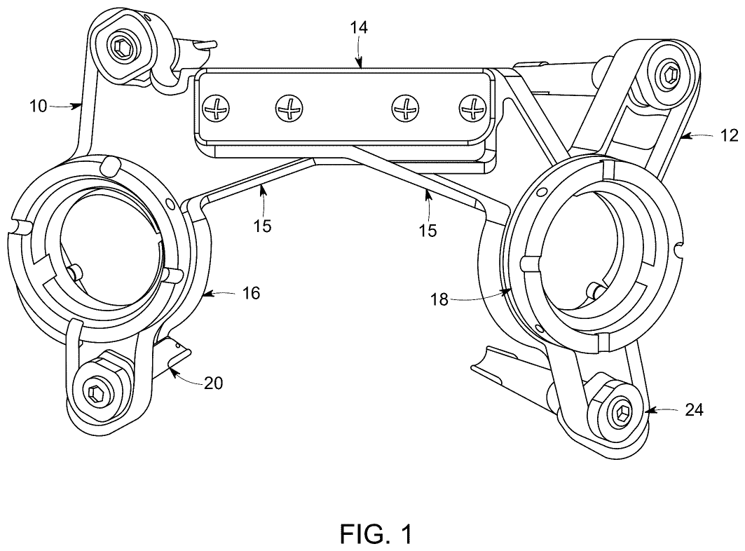

FIGS. 1-3 show top, side, and bottom views of the Bilateral surgical platform, which is composed of the following major pieces, each of which is depicted in these figures: two Bodies 10 and 12; one Connector 14 which is fastened to both Bodies via special bolts with spherical tops; two Couplers 16 and 18, each of which is inserted into a hole in one of the Bodies and attached to the Body with a Coupler Screw 19; and four Legs, each of which comprises a Shaft 20 with Threads 22, a Head 24, and two Grippers 26 for attachment to an anchor and is inserted into a hole in in one of the Bodies and attached with a Nut 28.

A key feature of the invention that is visible in FIGS. 1, 2, and 3 is that its design includes two Bodies 10 and 12. Two Bodies are included because manufacture of this invention is performed via the simplest, most accurate, and even cheapest machine for plastic pieces of specific shapes, a three-degree-of-freedom (“3DoF”) Computer-Numeric-Control router (“CNC”). A 3DoF CNC can provide two Bodies in order to guide two surgical instruments along two different trajectories with high accuracy. The two Bodies are connected together with a Connector 14, which is a flat metal blade that has been bent in the center at a specific angle. A set of Connectors of angles, e.g., 0, 10, 20, 30, and 40 degrees, are available from which a Connector of an optimal angle is selected for the assembly of a Platform of a required shape. The Connector is positioned relative to the Couplers, 16 and 18 so that when the Bilateral surgical platform is mounted onto the patient's head the Connector will be anterior to the line joining the centers of the Couplers. The purpose of this anterior positioning is to give the surgeon more access to the region of the head between the Couplers because the surgeon is typically located posteriorly to the head.

The reason for two Bodies to guide two instruments along different trajectories is as follows: Each guided instrument is fastened to one of two Couplers 16 and 18, and because the invention excludes all interoperative adjustments the guided instrument's trajectory is determined entirely by the orientation of the Coupler to which it is fastened. The orientation of each Coupler is determined by the orientation of the axis of the hole into which the Coupler is inserted, and that hole is cut with a 3DoF CNC. A 3DoF CNC can cut holes with axes perpendicular to the surface plane of the Body. Thus, the guided instrument's trajectory is determined by the surface plane of the Body. If the two Couplers were fastened to the same Body, the orientation of those Couplers and the trajectories of their two guided instruments would be identical, thus eliminating the attachment of two guiding of instruments along differing trajectories with one Body. Thus, the inclusion of two Bodies.

Two Bodies can provide differing trajectories for two guided instruments, by having differing orientations. Stated more specifically, the planar surface of one Body 10 should be perpendicular to the planned trajectory of one instrument, and the planar surface of the other Body 12 should be perpendicular to the planned trajectory of the other instrument. In addition to this invention's provision of two Bodies for guiding two instruments along different trajectories, it has a second, third, and fourth provision to permit these Bodies to have differing orientations. The second provision is the inclusion of a piece, called a Connector 14 that attaches the two Bodies together. The third provision is the placement of the Connector on the tops of Extended Sections 15 of each of the two Bodies. The top surface of each Extended Section is held in firm contact with the bottom surface of the Connector by means of Fasteners 30 and 32 for both bodies, which tighten the Connector to both Extended Sections with Fastener Nuts 34. The fourth provision, “stairsteps,” is further described below

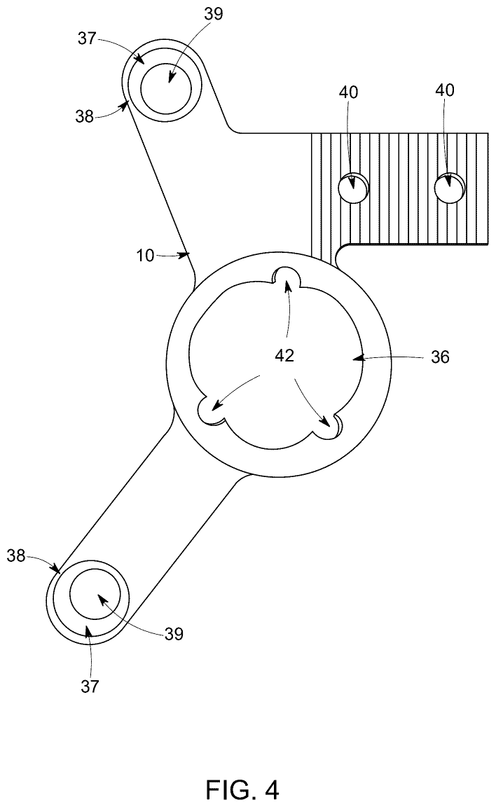

FIG. 4 shows one of the two Bodies with all attachments removed. The view is from the top, and there is no need for a view of the bottom or for a view of the top of the other body because both of these views come close to being mirror images. Each such view primarily shows the Body holes—a Coupler Hole 36, Leg Holes 38, Fastener Holes 40, and Coupler Screw Holes 42. Each Coupler Screw Hole intersects the Coupler Hole because of the size of the CNC drill bit in this example. If the drill bit is sufficiently small, the Coupler Screw Holes do not intersect with the Coupler Hole, but the Coupler is held sufficiently well with or without intersection. The Fastener Holes 40 are located properly to accommodate the slopes of the Fastener Connectors, which is a function of the relative orientation of the trajectories for the two guided surgical instruments.

In FIG. 4 an additional feature, Stairsteps, is visible and is further discussed below. Each Leg Hole 38 includes a Leg Countersink 38 at the top, which determines the horizontal position, the vertical position, and the orientation of the Leg Head 24, and a through-hole for the Leg Shaft 20. The depth of the countersink and the length of the Leg in combination determine the distance from the top of the Body to the bottom of the Leg. The shape of the Leg Countersink 37 matches the shape of the Leg Head 24, and its orientation also determines the orientation of the Grippers at the bottom of the Leg. A discussion of the importance of this orientation is given below.

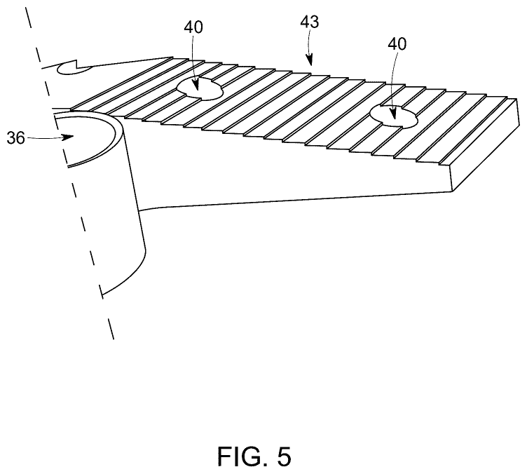

FIG. 5 shows the fourth provision of the invention for guiding two instruments along different planned trajectories—“Stairsteps” 43. Stairsteps, also referred to as “steps”, are a sequence of straight parallel cuts into the surface of a Body with each cut deeper than the previous cut by the same amount. Stairsteps are named after what they look like but are not in fact included for downstairs motion. Instead, they make it possible for a 3DoF CNC to shape the Bodies in a way that allows their planar surfaces to be oriented differently from each other. To accomplish the different orientations, Stairsteps are carved into the upper surface of each Body Extension 15 in order to cause the axis of the Coupler Hole 36 in that Body to be properly sloped relative to the Connector when that upper surface is held in firm contact with the bottom surface of the Connector. That contact is accomplished via the Fasteners 30 and 32 that pass through the Fastener Holes 40. If both Bodies are properly sloped relative to the Connector, then if the Platform is oriented so that the axis of one Coupler Hole 36 coincides with one of the planned trajectories, then the axis of the other Coupler Hole coincides with the axis of the other planned trajectory. The slope of the Stairsteps 43 will vary from case to case both in magnitude and direction. It is noted that in FIG. 5, in embodiments, the axes of holes 40 are perpendicular to the bottom surface of the stair steps portion.

In addition to the advantage of the two bodies being oriented so that the two Coupler axes can be parallel to the two trajectories instead of just one, the two legs attached to the body with the second coupler are considerably shortened by the fact that the top of each of those two legs is brought closer to the Extender to which they are attached than when there is only one planar top piece. Shortening a leg proportionally reduces the torque applied by it to the anchor to which it is attached when force is applied by the platform top perpendicularly to the leg. The typical angle—20 to 40 degrees—reduces the leg length and hence the torque applied to the anchor by 30% to 50%. Thus, any force communicated to the shorter legs will produce a 30% to 50% smaller torque on the bone anchors than when there is only one planar top piece. The reduction in torque on the anchors increases rigidity relative to the patient's head and hence increases accuracy when force is applied to the leg manually or gravitationally.

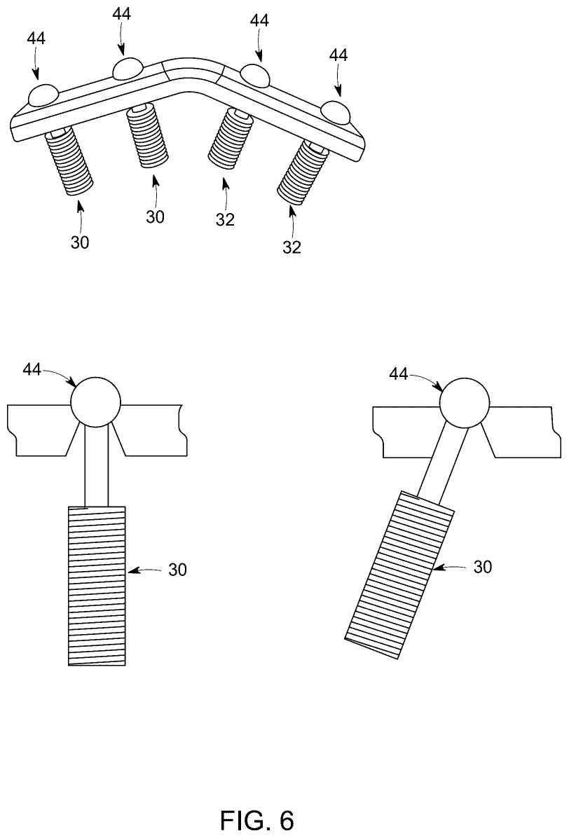

FIG. 6 shows a side-view and a bottom-view of a Connector that is not attached to a Body. The side-view shows Spherical Fastener Tops 44 and threaded bottoms of each Connector Fastener 30 and 32. The bottom views are cross sections of a Connector Screw Hole with a Fastener in the hole. As can be seen, the Screw Hole is partially hemispherical at the top and is conical at the bottom. The conical bottom portion allows the Connector Fasteners to be tilted when they are inserted into the Fastener Holes 40 in the Bodies. Each Spherical Fastener Top 44 sits in the partially hemispherical top portion of the hole. The hemispherical portion of the hole and the spherical top of the Spherical Fastener Top have the same radii, so that they serve together as a universal joint that maintains the connection between each Connector Fastener 30 and 32 and the Connector 14 to allow the Fastener to be tilted relative to the Connector.

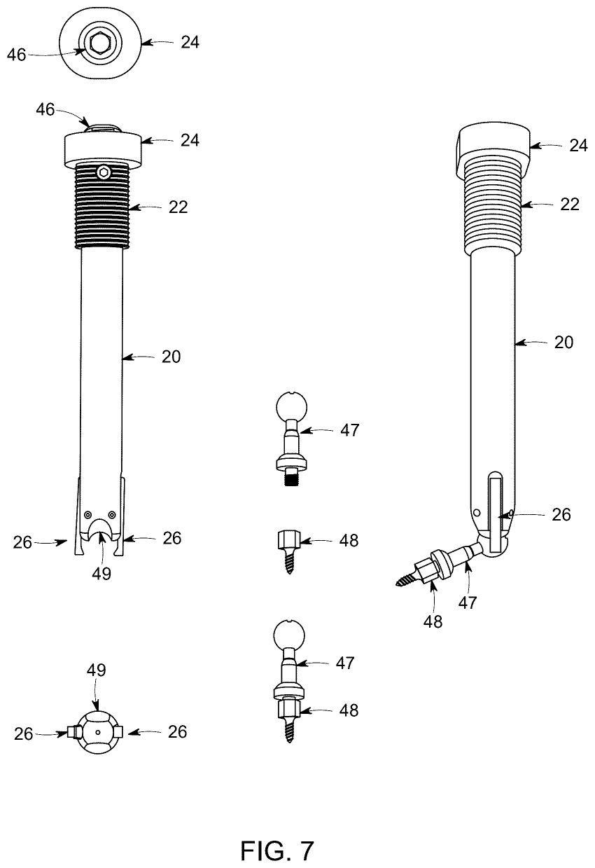

FIG. 7 shows the set of pieces involved in the mounting of the Bilateral surgical platform onto the anchors. The fundamental piece for mounting is the Leg, a side view of an example of which is shown on the left and right of the figure. At the top left of the figure a top view of the example leg is shown. This view shows the Leg Head 24, which has its length, which for this example is 75 mm, and its serial number engraved on top. In the center of the Leg Head is a hole, which descends through the Leg Shaft 20, and contains a Tightening Rod 46, whose purpose is described below. At the bottom left of the figure a bottom view of the Leg is shown. This view shows a Spherical Leg Indentation 49 and the tips of two Grippers 26 to the right and left of the indentation. The Spherical Leg Indentation 49 is also visible in the left side-view of the Leg between the Grippers 26 at the bottom of the Leg Shaft 20.

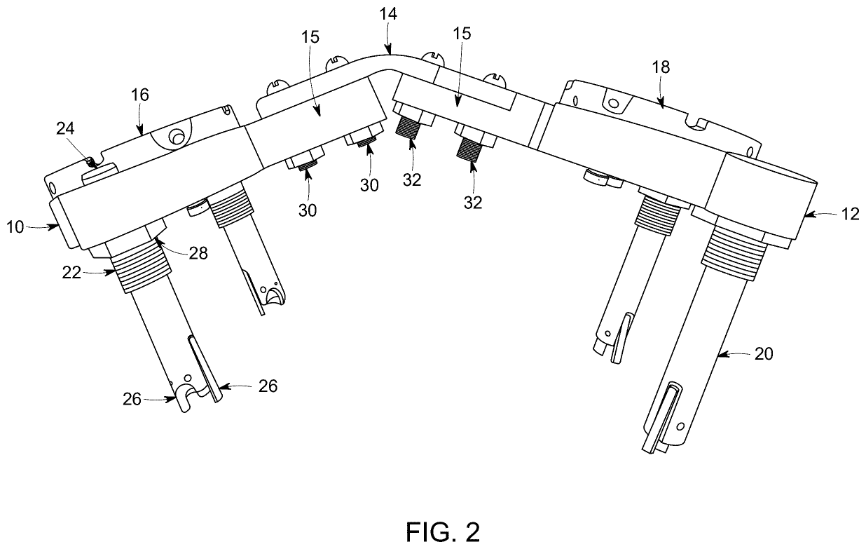

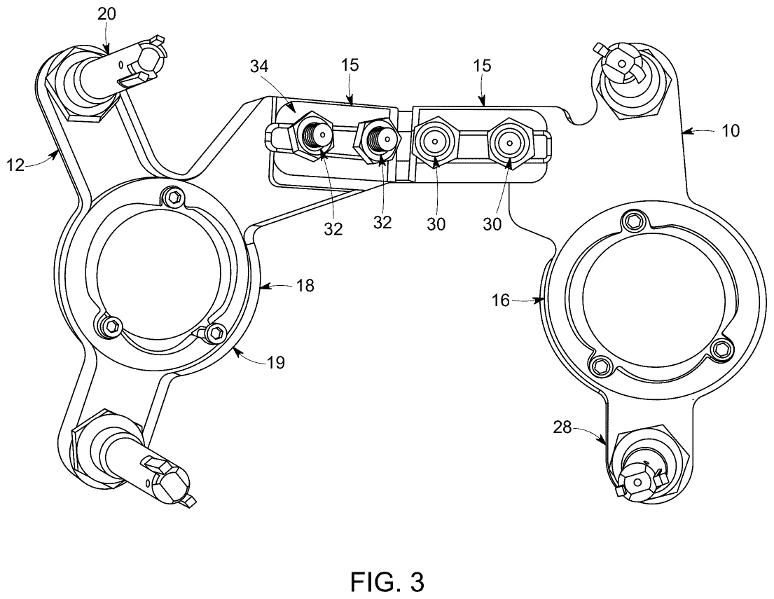

The following parts of the leg are involved in fastening the Leg to the Body in a Body Hole: the Leg Head 24, the Leg Shaft 20, and the Leg Threads 22. The Leg Head and a Leg Nut 28 (FIGS. 2 and 3) together put pressure on the top and bottom surfaces of the Body when the Leg Nut is tightened via the Leg Threads to sandwich the Body between with the Leg Shaft extending through the Leg Hole 38 (FIG. 4) toward the patient's head.

The following parts of the Leg are involved in fastening the Leg onto an Anchor 48: Grippers 26 at the bottom of the Leg and a Tightening Rod 46, which extends inside the Leg Shaft 20 from the Head 24 at the top of the Leg to the Grippers 26 at the bottom. In the operating room, an Extender 47 is hand screwed onto each bone-implanted Anchor after which the Spherical Leg Indentation 49 at the bottom of each Leg is set onto the spherical top of an Extender 47. Then, a hexagonal wrench is inserted into a hexagonal hole in the top of the Tightening Rod 46 and twisted. The twisting of the Tightening Rod tightens the Grippers 26 around the spherical top of the Extender 47 as shown at the bottom of the Leg on the right of this figure, thus rigidly mounting the bilateral surgical platform onto the patient's head. The angle of the Extender 47 relative to the Leg Shaft 20, is determined by the orientation of the skull-implanted Anchor 48, and the target trajectory that determines the orientation of the Body to which the Leg is attached, and that angle can be considerably greater than the typical angle shown in this figure without interference from the Grippers 26 because the Grippers are ideally oriented to avoid such interference. This ideal orientation is achieved by the orientation of the Leg Countersink, as discussed above.

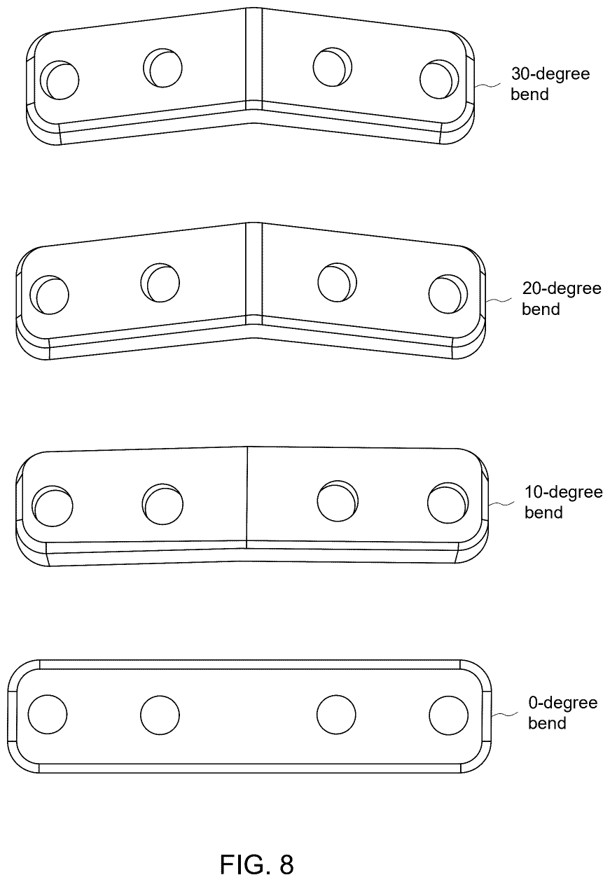

FIG. 8 shows alternatives of the Connector's bend angle from the 40-degree angle in FIGS. 1, 2, 3, and 6. The bend angle is the deviation between the planar surfaces of the left and right sections of the Connector. The purpose of bending the Connector is to reduce the necessary angle of the required slants of the Stairsteps in each of the two Body Extensions Connectors in order to orient the two sections of the top, thus, increasing the mean thickness of the Extensions where the Stairsteps are cut and, hence, increasing stiffness. When a Connector with a bend angle of X degrees is used instead of 0 degrees, the necessary slope of the Stairsteps in each of the two Body Extensions is reduced by X/2 degrees compared to the necessary slope with a 0-degree Connector. The ideal slopes of the two Stairsteps 43 are equal to each other because making them equal minimizes the maximum slope. These slopes can be in any direction, but the component of those slopes parallel to the longest edge of the Connector, is always much larger than the component perpendicular to that edge. After the angle A of that component of the ideal slope for the flat Connector is calculated, an Angled Connector 58 can be chosen from among an available set of Angled Connectors 58 with an angle Y that is not greater than 2A. The resulting component in that direction of the required Stairsteps slope for each Body for that Angled Connector is A-Y/2. This reduction in slope by Y/2 improves the connection between the Bodies by increasing the mean thickness of the Extension. As an example, the dimensions of a connector can be about 85 mm in length and about 19 mm in width with a depth of about 6 mm.

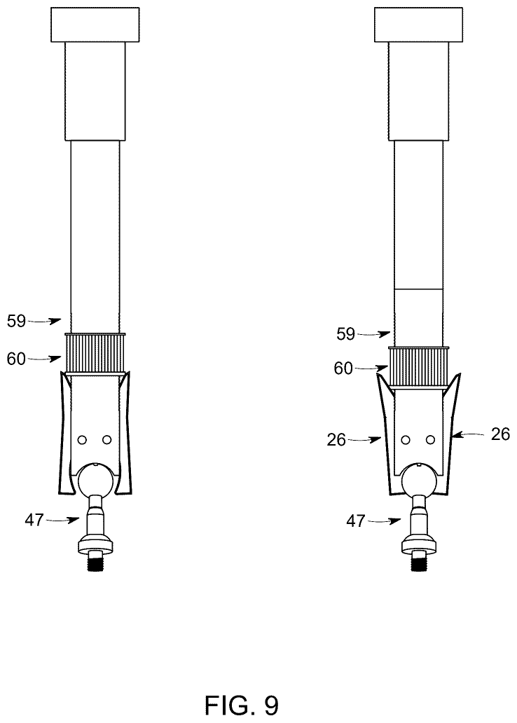

FIG. 9 shows an alternative embodiment of a Leg design. In this embodiment the Leg is the same as that of FIG. 7 except that the Tightening Rod is omitted and the Grippers 26 are pushed together by a Gripper Tightener 60. The Gripper Tightener is a hollow cylinder with female internal threads that fit external male threads 59 on the Leg near the bottom, which when the Tightener is twisted moves it toward or away from the Grippers. On the left is shown the alternative Leg with the Gripper Tightener 60 above the Grippers 26 and the spherical indentation at the bottom of the Leg just above an Extender 47. On the right the alternative Leg with its Gripper Tightener 60 has been screwed down against the upper sections of the Grippers so that they are pushed out at the top. Pushing them out at the top forces them in at the bottom because they are each attached to the Leg via an axle pin, each of which is illustrated in the figure with a black circular dot. As can be seen in the right side of the figure, when the Grippers 26 are pushed together the Leg is firmly to Extender 47.

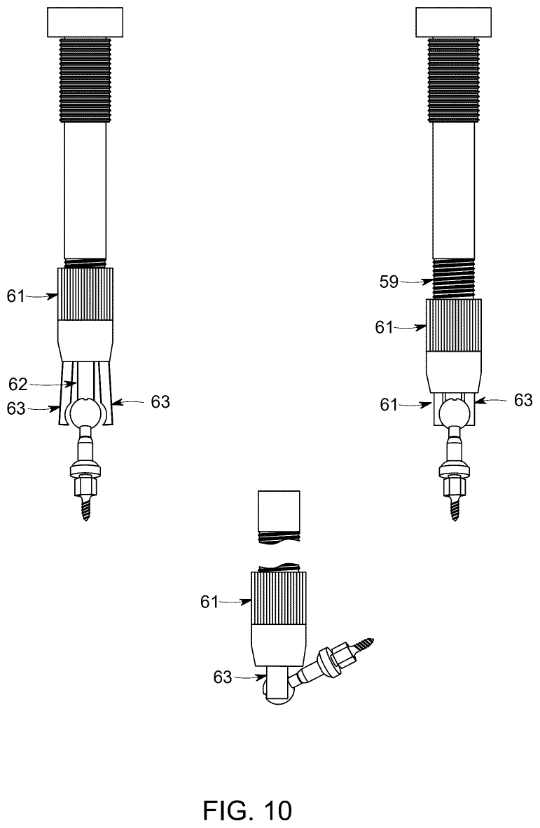

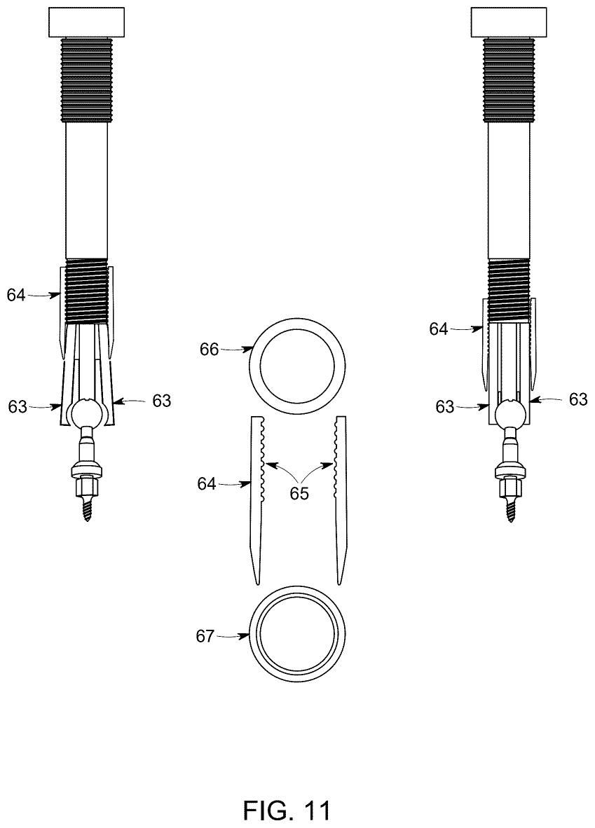

FIGS. 10 and 11 show an alternative embodiment of the Leg design. In this embodiment, the Leg is the same as that of FIG. 7 except that the Tightening Rod and the Grippers are omitted, the end of the Leg is split into a middle part 62 and two outer parts 63, and the outer parts are partially inside a Leg-end Tightener 61. The Leg-end Tightener is a hollow cylinder with female internal threads that fit external male threads 59 on the Leg near the bottom, which when the Tightener is twisted in one direction or the other moves it toward or away from the end of the split end of the Leg. On the left side of each figure is shown the alternative Leg with the Tightener 61 far enough above the end of the Leg that the outer parts of the leg-split are in their natural outspread positions which are far enough apart to allow the end of the Leg to be placed on the spherical end of an Extender 47. As shown clearly on the left side, a spherical indentation at the bottom of the middle part 62 of the leg-split fits onto the spherical end of the Extender 47. On the right side the Leg-end Tightener 61 has been screwed down over the outer split-ends tightening them against spherical end of the Extender 47. As can be seen in the right side of the figure, when the outer parts of the leg-split are pushed together the Leg is firmly attached to Extender 47. The black parts shown on the left and right, and in the middle are cross sections of the Leg-end Tightener through two planes: 64 in an axial plane, 65 in the same axial plane showing the Tightener's threads, 66 in plane perpendicular to the axis at the top of the Tightener, and 67 in plane perpendicular to the axis at the bottom of the Tightener.

For reference the labels used in the figures are as follows. 10: Body-1; 12: Body-2; 14: Connector; 15: Body Extension; 16: Coupler-1; 18: Coupler-2; 19: Coupler Screw; 20: Leg Shaft; 22: Leg Threads; 24: Leg Head; 26: Gripper; 28: Leg Nut; 30: Connector Fastener for Body-1; 32: Connector Fastener for Body-2; 34: Connector Fastener Nut; 36: Coupler Hole; 37: Leg Countersink; 38: Leg Hole; 39: Leg Through-Hole; 40: Fastener Hole; 42: Coupler Screw Hole; 43: Stairsteps; 44: Spherical Fastener Top; 45: Connector Screw Holes; 46: Tightening Rod; 47: Extender; 48: Anchor; 49: Spherical Leg Indentation; 50: Connector Cover; 52: Connector Cover Screw Holes; 54: Connector Cover Screws; 56: Connector Cover Spherical Divots; 58: Angled Connector; 59: External male threads for Gripper Tightener; 60: Gripper Tightener with internal female threads; 61: Leg-end Tightener with internal female threads; 62: Middle section of split end of the Leg; 63: Outer section of split end of the Leg; 64: Cross-section of 61 through an axial plane; 65: Cross-section of 61 through an axial plane showing threads; 66: Cross-section of 61 at top through a plane perpendicular to its axis; 67: Cross-section of 61 at the bottom through a plane perpendicular to its axis.

### EXAMPLES

Each bilateral surgical platform may be customized to be patient specific. To customize a bilateral surgical platform for a specific surgical case, a surgeon runs a computer program, typically on a computer in the hospital. That program can be a Surgical Planner for surgical-instrument guidance into patient anatomy. The planner loads a CT image volume of a patient's head, which was acquired after four Anchors were implanted in the skull. With the help of the planner, the surgeon then determines in the coordinate system of a Computed Tomography Scan (CT) volume the position and orientation of each of the Anchors, the entry point and target point for each trajectory, the positions of the anterior commissure, the posterior commissure, and a midplane point and stipulates the distance from a standard point on the surgical instrument to the target point. A midplane point is any point that lies on the midsagittal plane of the brain, which is the plane that divides the brain into left and right hemispheres. This information is then saved in a Planning File, which is then sent electronically to a platform-manufacturing location.

The Planning File generated by the Surgical Planner is read by a Manufacturing Program on a computer at a platform-manufacturing location. The Manufacturing Program, based on the information in the Planning File, generates a CNC Program, which is a list of instructions in a standard CNC language called Gcode™. The purpose of the CNC Program is to manipulate a CNC to cut out the two Bodies of the Bilateral surgical platform from a planar blank. The Manufacturing Program also issues a list of Leg lengths for the Legs that will be attached to those Bodies and a bend angle for the connector. The shapes of the Bodies, the positions of the holes in the Bodies, and the depths of the Leg Countersinks are all incorporated in the CNC program.

The CNC program generated by the Manufacturing Program is loaded into a CNC's digital memory. A planar blank is rigidly secured onto the CNC's router table with the CNC's cutting bit set above the blank. The CNC program is then run by the CNC, and the CNC cutting bit begins to spin, moves down to the blank and begins cutting into it. It cuts two Bodies 10 and 12 from the blank, cuts the Stairsteps 43 and the countersinks 37 and holes 38, 39, 40, 42, and 45 and then the bit rises above the Bodies and turns off. The Bilateral surgical platform is then manually assembled from the Bodies 10 and 12, a Connector 14, Legs 20, 22, 24, 26, 46 of the specified lengths, and Couplers 16 and 18 with Leg Nuts 28, Connector Fastener Nuts 34, and Coupler Screws 19. The resulting assembly positions and orients each Coupler relative to the spherical indentations at the bottom of the Legs so that when these indentations are placed on the spherical tops of Extenders that have been screwed into the Anchors and surgical instruments have been attached to the Couplers, each instrument will be aimed at its target along the trajectory from the entry point to the target point. Alternatively two CNC programs can be generated by the Manufacturing Program and one loaded into one CNC's digital memory and other loaded into a second CNC's digital memory; two planar blanks can be rigidly secured, one onto each CNC's router table; each CNC program can be run by one of the CNC; Body 10 can be cut, as described above, on one CNC; and Body 11 can be cut, as described above, cut as described above on the other CNC.

The preferred thickness of a blank from which the Bodies are carved by the CNC is 12.7 mm. The preferred lengths of the Legs equal 15+10 n mm, where n is a positive integer less than 11. However, if the Body thickness is smaller than 12.7 mm the 10 in the formula must be replaced by a smaller number. As shown in FIG. 8, the preferred set of angles for the Connector's bend are 0, 10, 20, 30, and 40 degrees and its preferred linear dimensions are length=85 mm, width=19 mm, and depth=6 mm. The length is measured along the regardless of the bend angle. Thus, the distance between the ends is smaller for a larger bend: distance=l cos(θ/2), where l=length, e.g., for an angle of 40 degrees the distance is 79.87 mm. The preferred length, thread diameter, and spherical diameter of the Connector Fasteners are 32, 7, and 6 mm. The preferred diameters of the Leg Shaft threads are 9 and 11 mm. The preferred dimensions of the Leg Heads are a thickness of 6 mm and any non-circular shape for countersinking into a Body that fits within a circle of 16 mm. The preferred outer diameter, inner diameter, and thickness of the Coupler are 50, 32, and 14 mm. The preferred depth of each Stairstep is one millimeter. The dimensions are all approximate.

In some cases, the bilateral surgical platform may be used to guide just one surgical instrument along one trajectory toward one target. In this case the Surgical Planner will produce a Planning File that has only one entry point and one target point. Based on this Planning File, the Manufacturing Program will generate a mock second entry point and a mock second target point so that both Bodies can be generated—one for the planned trajectory and one for a mock trajectory. A mock point can be generated in multiple ways. For example, it can be positioned symmetrically to the actual point relative to the midplane. Furthermore, the Body with the mock trajectory can be cut differently from the usual Body by omitting its Coupler Hole 36 and reducing the size of the circular portion of the Body where the Coupler Hole would have been.

In some cases, only three Anchors may be implanted in the patient's skull. In this case the Surgical Planner will produce a Planning File that has a position and orientation for only three anchors, regardless of whether there are one or two targets. In this case based on this Planning File, the Manufacturing Program will generate one Body with two Leg Holes, and one Body with one Leg Hole. For example, the Bilateral surgical platform could require for the Body to the left of midplane a left-anterior Leg of length 65 mm and a left-posterior Leg of length 55 mm and for the Body to the right only one Leg of 75 mm.

A major advantage of the Bilateral surgical platform is that it can be made with the simplest, smallest, lightest, cheapest, and most accurate type of CNC—the 3DoF CNC. However, an obvious alternative is to use a CNC with more than three degrees of freedom. If the CNC has four or five degrees of freedom there are multiple changes that can be made in the design, but a preferred key change is that Stairsteps 43 would not be necessary to produce the required slants of the upper surfaces of the two Body Extensions 15. Instead, the upper surfaces could be carved smoothly with proper slopes.

### GLOSSARY

A planar body, as used herein, refers to a body with a planar section and a section that is angled with respect to planar section. An Example is shown in FIG. 4. In FIG. 4, the planar section is the section on the left side including the two wings and the angled section includes holes 40. The angled section is more clearly shown in FIG. 5.

A connector, as used herein, is a component with a bend at or near its center that forms an angle for the connector as a whole. Examples are shown in FIG. 8 with different angled degrees of bend. In embodiments, the angled section of the planar body referred to above is angled to correspond with, or match, the angle of the bend of the connector.

A coupler, as used herein, refers to a component that couples a surgical instrument with the planar body and aligns the surgical instrument at a given, or predetermined, trajectory. The coupler is disposed in a hole in the planar body. Examples of couplers are shown in FIGS. 1-3.

Trajectory, as used herein, refers to a straight line joining an entry point and a target point along the planned path of a surgical probe. Generally speaking, while the surgeon plans the path in reference to the CT coordinates of the patient's anatomy, the planned path for the purposes of the patent is relative to the bilateral surgical platform itself, regardless of whether the bilateral surgical platform has yet been attached to the patient.

A midplane point, as used herein, is any point that lies on the midsagittal plane of the brain, which is the plane that divides the brain into left and right hemispheres.

While this disclosure includes specific examples, it will be apparent after an understanding of the disclosure of this application has been attained that various changes in form and details may be made in these examples without departing from the spirit and scope of the claims and their equivalents.

1. A bilateral surgical platform that is configured to simultaneously align two surgical instruments along different trajectories during surgery comprising:
two planar bodies, wherein each planar body comprises a planar portion and an angled portion that forms an angle with the planar portion, and a hole;
two couplers each for holding a surgical instrument aligned at a predetermined trajectory, wherein one coupler is inserted into the hole in one of the planar bodies and the other coupler is inserted into the hole in the other planar body;
a connector, wherein the connector is attached to each planar body and has a bend forming an angle at or near its center, wherein the connector is attached to the angled portion of the planar bodies, and the angle of the connector and the angle of the angled portion of the planar bodies are configured to align the surgical instrument(s) at the predetermined trajectory; and
further wherein each planar body comprises one or several legs, with each leg configured to attach to an anchor implanted in a patient's head.

2. The bilateral surgical platform of claim 1, wherein there are two surgical instruments and the planar portion of one planar body is perpendicular to the predetermined trajectory of one surgical instrument, and the planar portion of the other planar body is perpendicular to the predetermined trajectory of the second surgical instrument.

3. The bilateral surgical platform of claim 1, wherein the two planar bodies comprise a thermoplastic polymer or a metal, the two couplers comprise a thermoplastic polymer or a metal, the connector comprises a thermoplastic polymer or a metal, and each leg comprises a thermoplastic polymer or a metal.

4. The bilateral surgical platform of claim 1, wherein the two planar bodies comprise polycarbonate, polyetherimide, polyether ether ketone, aluminum, stainless steel, or titanium; the two couplers comprise polycarbonate, polyetherimide, polyether ether ketone, aluminum, stainless steel, or titanium; the connector comprises polycarbonate, polyetherimide, polyether ether ketone, aluminum, stainless steel, or titanium; and each leg comprises polycarbonate, polyetherimide, polyether ether ketone, aluminum, stainless steel, or titanium.

5. The bilateral surgical platform of claim 1, wherein the two planar bodies comprise polycarbonate; the two couplers comprise polycarbonate; the connector comprises aluminum; and each leg comprises stainless steel or titanium.

6. The bilateral surgical platform of claim 1, wherein the anchor comprises titanium.

7. The bilateral surgical platform of claim 1, wherein each planar body comprises two legs.

8. The bilateral surgical platform of claim 1, wherein one planar body comprises one leg and the other planar body comprises two legs.

9. A method of making the bilateral surgical platform of claim 1, comprising:
implanting a plurality of anchors into a patient's head;
taking a Computed Tomography Scan of a patient's head including the anchors, wherein the Computed Tomography Scan comprises a coordinate system;
determining coordinates for the position and orientation for each anchor, for each entry point and target point on the patient's head, the anterior commissure, the posterior commissure, and a midplane point;
determining, using the coordinates, with a manufacturing computer program, the shapes of the two planar bodies, the positions of the holes in the two planar bodies, and the lengths of the legs;
manufacturing the planar bodies; and
manually assembling the bilateral surgical platform.

10. The method of claim 9, wherein the computer program comprises a three-degree-of-freedom Computer-Numeric-Control (CNC) router.

11. The method of claim 9, wherein the manufacturing of the angled portion of the planar bodies comprises forming a set of steps comprising straight parallel cuts into the surface of the planar body with each cut deeper than the previous cut by the same amount.
